(19) United States
(12) Patent Application Publication
(10) Pub. No.: US 2008/0044398 A1
(43) Pub. Date: Feb. 21, 2008

(54) Soluble composition having enzymatic activity and improved stability

(75) Inventor: Sangyeol Kwon, Indianapolis, IN (US)

Correspondence Address: VESTA INGREDIENTS, INC., 5767 THUNDERBIRD RD., INDIANAPLIS, IN, 46236, US

(73) Assignee: Vesta Ingredients, Inc., Indianapolis, IN (US)

(21) Appl. No.: 11/486,419

(22) Filed: Jul. 13, 2006

(51) Int. Cl. A23C 21/00; A23L 1/30; A23L 2/00; A61K 38/54

(52) U.S. Cl. 424/94.3; 426/583; 426/599; 426/648

(57) ABSTRACT

A dry, powdered enzyme is dispersed in a dry, powdered maltodextrin carrier by dry mixing to provide a soluble composition having enzymatic activity and improved stability. The composition provides fibrinolytic activity when the enzyme is a fibrinolytic enzyme, such as nattokinase, and the fibrinolytic activity is added to supplement foods when the composition is dissolved in liquid food components.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

This invention relates generally to soluble compositions having enzymatic activity and more particularly to soluble compositions having fibrinolytic activity and improved stability, to supplement foods containing such compositions and to the treatment of subjects by the ingestion of such supplement foods.

2. Background

Nattokinase is known to have measurable fibrinolytic and thrombolytic activity. Nattokinase was first described in 1987 as an extract from Natto, or fermented soy beans. Natto itself has a long history. By some accounts it has been a staple in parts of Japan since the Heian era (794 to 1192) and has long been understood to have exceptional health benefits. The 1987 publication described nattokinase as associated with the reduction of blood clots in dogs, and since 1987 a thriving and still-growing industry has developed in supplying nattokinase for use in supplement foods.

In commercial applications, nattokinase is extracted from fermented natto and is purified by sequential hydrophobic chromatography. Like most enzymes, nattokinase is protected from light and ambient humidity during production and storage. Nattokinase is normally produced and stored at temperatures below about 40° C. The fibrinolytic activity of the extracted and purified nattokinase is measured by the method described by Hsien-Yi Sung et al. in J. Agric. Food Chem. 2000, 48, 3210-3216 (hereinafter “the Hsien-Yi Sung assay”). The Hsien-Yi Sung assay reports fibrinolytic activity in Fibrinolytic Units per gram (FU/g). Each batch of nattokinase may have an individual level of fibrinolytic activity, as would be expected from a fermentation process.

Nattokinase enzyme typically is mixed with a carrier prior to being placed in final form for end users in order to dilute and to standardize its fibrinolytic activity. Extracted nattokinase may have a fibrinolytic activity level in the range of 260,000 FU/g, while the desired level for use in capsules and tablets as nutritional supplements is typically about 20,000 FU/g. The ratio of the enzyme to carrier can be adjusted to achieve a desired, pre-determined enzyme activity level for the enzyme-carrier composition, compensating for variations in the activity level of the extracted and purified enzyme. Starch and microcrystalline cellulose (MCC) carriers have been used with success in the production of tablets and capsules having nattokinase activity. Riordan, in U.S. application 2004/0223962 A1, describes a nattokinase-antioxidant combination for use as a nutritional supplement delivered by means of capsules that are filled with the combination and with excipients to adjust the fibrinolytic activity delivered by the combination. Dextrins are described by Riordan as useful carbohydrate excipients for use in capsules.

Starch and MCC carriers of high quality for nattokinase enzyme are relatively inexpensive and readily available. Both starch and MCC carriers are well known and generally accepted for use with enzymes when filling capsules or when making tablets. However, both starch and MCC carriers have demonstrated problems when attempts were made to add enzyme activity to foods by dissolving the enzyme/carrier mixture in liquids such as milk, water, yogurt and fruit juices. MCC is essentially insoluble, and starch carriers can form clumps that are difficult to disperse. There has been a need in the industry for a carrier for enzymes such as nattokinase that will facilitate adding enzyme activity to liquids for use in place of capsules and tablets as supplement foods.

Nattokinase, like most enzymes, tends to lose its activity over time, even during careful storage where the enzyme is protected from light, heat and moisture. The industry is continually searching for methods or materials to improve stability, or shelf life, of biological materials, including enzymes and compositions having enzyme activity. For example, Franks, et al. disclose in U.S. Pat. No. 5,928,469, in U.S. Pat. No. 6,825,031 B2 and in U.S. Application 2005/0106553 improving the stability of Lactobacillus by adding Lactobacillus cells to a solution of maltodextrin and spray drying the liquid to produce Lactobacillus held in a glassy, amorphous or rubbery substance. Barendese, et al. describe in U.S. Pat. No. 6,500,426 B2 enhancing the storage stability of edible enzymes useful, for example, especially in livestock feed by forming a mixture of the enzyme with a starch and extruding the mixture to form edible pellets that contain and protect the enzyme.

In U.S. Pat. No. 6,677,141 B2 and U.S. Pat. No. 6,677,143 B2 Sumi teaches roasted dextrin as a carbon source for use by Bacillus subtilis in the production of vitamin K (MK-7) showing photostability. Moriyama, et al. teach in U.S. Application 2004/0043015 A1 the use of dextrin powder in combination with a sucrose fatty acid ester as a filler material when adding a Bacillus natto culture extract to capsules. In U.S. Application 2004/0209333 Takaoka discloses forming a paste of a Bacillus natto culture from which most Vitamin K activity has been removed, adding starches, sugars, or cellulose to the paste and freeze drying the paste to form a powder from which tablets may be pressed or with which capsules may be filled. Moriyama, et al., in U.S. Application 2005/0025759 A1, describes the use of Bacillus natto culture powder as a supplement food in capsules, sometimes mixed with materials such as soybean lecithin, soybean oil, beeswax, glycerin and gelatin.

None of these publications show or suggest a soluble, dry, powdered composition comprising a dry, powdered enzyme dispersed in a dry, powdered maltodextrin wherein the composition has nattokinase or other enzymatic activity and enhanced stability. Further, none of these publications show or suggest an edible or drinkable supplement food that includes such a composition in solution.

### BRIEF SUMMARY OF THE INVENTION

It is one object of this invention to fill the need for a liquid-soluble composition having enzymatic activity, especially nattokinase activity.

It is another object of this invention to fill the need for improved stability in compositions having enzymatic activity, especially in compositions having nattokinase activity.

It is another object of this invention to provide a method for making such a composition.

It is still another object of the present invention to provide liquid supplement foods that include a composition having enzymatic activity, especially nattokinase activity.

These and other objects are achieved by the present invention which, in one aspect, is a stable composition that is soluble in liquid food components and that has enzymatic activity, the composition comprising dry, powdered enzyme dispersed in a dry, powdered maltodextrin carrier material.

In another aspect, the invention is a method of making a stable, dry, powdered composition having enzymatic activity and solubility in liquid food components, the method comprising the steps of providing a dry, powdered enzyme and dispersing the enzyme by dry mixing in a dry, powdered maltodextrin carrier material.

In still another aspect, the invention is a supplement food having enzymatic activity comprising a dry, powdered composition having enzymatic activity dissolved in a liquid component of the supplement food, the powdered composition comprising an enzyme dispersed in a dry, powdered maltodextrin carrier material.

### BRIEF DESCRIPTION OF THE SEVERAL VIEWS OF THE DRAWING

FIG. 1 shows stability of the composition of the present invention compared with a composition made using a lactose carrier material and with the enzyme alone.

FIG. 2 shows the comparative stability of the same three materials as shown in FIG. 1 but at higher temperature and humidity.

### DETAILED DESCRIPTION OF THE INVENTION

Any dry, powdered enzyme may be used in the composition of the present invention. An enzyme that provides fibrinolytic activity is preferred. Good results have been achieved using nattokinase, which has fibrinolytic activity, and the invention will be exemplified with nattokinase, although it will be understood by those familiar with the art that other enzymes will be useful, especially other enzymes that provide fibrinolytic activity such as Bacillopeptidase F described in US pending patent application 2006/0105071.

Nattokinase is now readily available from a variety of suppliers who extract it from a natto fermentation and purify it, usually by chromatography. Because nattokinase is produced by fermentation, each batch can be expected to have an individual level of fibrinolytic activity. Fibrinolytic activity of nattokinase is typically measured by the Hsien-Yi Sung assay.

Most manufacturers of nutraceuticals and supplement foods prefer a composition having a fibrinolytic activity of about 20,000 FU/g. The purified enzyme typically has fibrinolytic activity in the range of about 260,000 FU/g. This high activity is usually adjusted to the desired level (normally about 20,000 FU/g) by mixing it with a carrier such as MCC, starch or lactose powder. The ratio of the enzyme to the carrier material can be adjusted to achieve a pre-determined target enzyme activity level.

Surprisingly, it has been discovered that dispersing the dry, powdered enzyme in an appropriate amount of dry, powdered maltodextrin carrier material not only achieves the desired enzyme activity level but also dramatically improves the shelf life of the composition. Further, the composition was found to be readily soluble in liquid components of supplement foods such as milk, yogurt, water, and fruit and vegetable juices.

The desired FU/g activity level of the composition of the present invention is usually achieved with a ratio of nattokinase enzyme to maltodextrin of about 20%, although the ratio typically will vary depending on the activity of each particular batch of nattokinase enzyme. The nattokinase enzyme is normally supplied in dry, powder form with an average particle size of about 40 mesh.

The nattokinase enzyme is dispersed in a dry, powdered maltodextrin carrier material. Any source of such maltodextrin may be used. Good results have been obtained with maltodextrin from Tate & Lyle, London, Tenn. The average mesh size of the maltodextrin obtained from Tate & Lyle was about 60.

Those familiar with powders will understand that mesh sizes will vary as a measurement of particle size depending on the gauge of the wire with which a mesh is woven. For example, commercial mesh suppliers describe a 40 mesh screen as relating to particle sizes of from 381 to 410 micron diameter, and a 60 mesh screen is described as corresponding to particle sizes of from 229 to 260 microns.

Dry powder, as used in the present invention, is intended to mean a powder that is sufficiently free of moisture as to clumping or aggregation and as to retain fluid characteristics. Dry powdered materials are generally understood to include less than about 5% moisture or liquid content.

The enzyme powder is dispersed in the maltodextrin by any useful type of dry mixing at relatively low temperatures (below about 40° C.). Good results have been obtained by mixing the enzyme powder and the maltodextrin carrier material in a ribbon mill. Mixing is accomplished without adding liquid to either dry powder. Mixing times are typically about 20 minutes to achieve substantially uniform dispersion of the enzyme in the carrier material.

Surprisingly, such compositions have been found to demonstrate improved stability or shelf life during both normal and accelerated storage. Compositions of the present invention were found to have improved stability characteristics when compared with dry powder mixtures of enzyme and lactose, which is also a sugar. Unlike compositions that include enzyme with starch or MCC carriers, the enzyme/maltodextrin composition has been found to readily dissolve in liquid food components such as water, yogurt, milk, sauces such as apple sauce, and fruit and vegetable juices. This solubility facilitates the addition of enzymatic activity such as fibrinolytic activity to ingestible supplement foods, expanding the commercially useful field of such supplement foods beyond capsules and tablets.

Ingestible supplement foods according to the present invention have been made by dissolving a selected amount of a dry powder comprising a dry, powdered enzyme dispersed by dry mixing in a dry, powdered maltodextrin material in a liquid food component. The selected amount may be sufficient to give the supplement food the desired enzymatic activity, such as a fibrinolytic activity. The preferred supplement food according to the present invention includes nattokinase to provide fibrinolytic activity.

Supplement foods are used to treat subjects by adding enzymatic activity, such as fibrinolytic activity, to the subject by ingestion. The amount of enzymatic activity that is added to the subject will vary from subject to subject.

### EXAMPLE

Nattokinase enzyme, which is commercially available from a variety of well known sources, was obtained in dry, powdered form from a Korean contract manufacturer. The enzyme was reported by the manufacturer to have an activity of 265,000 FU/g and a particle size of 60 mesh. Six separate 100 g portions of the enzyme power were isolated from the bulk supply. Five grams from each of two of the six portions was assayed by the Hsien-Yi Sung assay to confirm the reported fibrinolytic activity and the balance of the two portions were sealed in air-tight high density polyethylene (HDPE) pharmaceutical bottles. Two of the four remaining portions were dispersed in 1.3 kg of dry, powdered lactose having a particle size of 60 mesh by processing in a ribbon mill for 20 minutes. A 5 g portion from each of the two lactose dispersions was tested by the Hsien-Yi Sung assay to determine its fibrinolytic activity, and the balance of each lactose dispersion was placed in an HDPE pharmaceutical bottle. The enzymatic activity of the lactose dispersions was 20,100 FU/g.

The remaining two separated portions of enzyme were dispersed in 1.3 kg of dry, powdered maltodextrin having a particle size of 60 mesh. The enzyme/maltodextrin dispersion also was accomplished by processing in a ribbon mill for 20 minutes. A 5 g sample from each of the two maltodextrin dispersions was measured by the Hsien-Yi Sung assay to determine its fibronilytic activity, and the balance of each maltodextrin dispersion was placed in an air-tight HDPE pharmaceutical bottle. The enzymatic activity of the maltodextrin dispersions was 20,100 FU/g.

One air-tight HDPE pharmaceutical bottle each of the maltodextrin dispersions, the lactose dispersions and the undispersed nattokinase samples was placed in a standard storage room having controlled temperature of 25° C. (+or −2° C.) and having controlled humidity of 65% (+or −5%). Additional 5 g samples of the lactose/enzyme dispersion and of the maltodextrin/enzyme dispersion were taken from each of the air-tight HDPE pharmaceutical bottles kept in the standard storage room and assayed for changes in fibrinolytic activity on days 63, 120, 152, 183, 150, 343, 434 and 526. The assay results are shown in Table 1.

Five gram samples of the undispersed enzyme kept in the standard storage room were taken from the air-tight HDPE pharmaceutical bottle in which it was held and assayed for changes in fibrinolytic activity on days 21, 99, 156, 219, 286, 379, 470 and 562. The assay results are shown in Table 2.

One air-tight HDPE pharmaceutical bottle each of the maltodextrin dispersions, the lactose dispersions and the undispersed nattokinase samples was placed in an environmental test chamber for accelerated stability testing at a temperature of 40° C. (+or −2° C.) and a humidity of 75% (+or −5%). Additional 5 g samples were taken from each of the air-tight HDPE pharmaceutical bottles in the environmental test chamber and assayed for changes in fibrinolytic activity on days 50, 107, 139, 170, 237 and 330. The assay results are shown in Table 3.

Five gram samples of the undispersed enzyme kept in the environmental test chamber were taken from the air-tight HDPE pharmaceutical bottle in which it was held and assayed for changes in fibrinolytic activity on days 27, 84, 116, 147, 214 and 304. The assay results are shown in Table 4.

TABLE 1
Dispersed Enzyme in Standard Storage Room

| | | Lactose | Maltodextrin | Maltodextrin |
|---|---|---|---|---|
| | Lactose | dispersion | dispersion | dispersion |
| Duration | dispersion | relative activity | activity | relative |
| (Days) | activity (FU/g) | (%) | (FU/g) | activity (%) |
| 0 | 20,100 | 100 | 20,100 | 100 |
| 63 | 19,367 | 96 | 20,300 | 101 |
| 120 | 19,095 | 95 | 20,967 | 104 |
| 152 | 18,967 | 94 | 21,233 | 106 |
| 183 | 19,533 | 97 | 20,733 | 103 |
| 250 | 17,733 | 88 | 21,067 | 105 |
| 343 | 18,559 | 92 | 20,390 | 101 |
| 434 | 16,727 | 83 | 19,121 | 95 |

TABLE 2
Undispersed Enzyme in Standard Storage Room

| Duration | Enzyme | Relative |
|---|---|---|
| (Days) | Activity (FU/g) | Enzyme Activity (%) |
| 0 | 265,000 | 100 |
| 21 | 227,667 | 86 |
| 99 | 210,667 | 79 |
| 156 | 205,667 | 78 |
| 219 | 201,333 | 76 |
| 286 | 201,555 | 76 |
| 379 | 199,700 | 75 |
| 470 | 195,450 | 74 |

TABLE 3
Dispersed Enzyme in Environmental Test Chamber

| | | Lactose | | Maltodextrin |
|---|---|---|---|---|
| | Lactose | Dispersion | Maltodextrin | Dispersion |
| Duration | Dispersion | Relative | Dispersion | Relative |
| (Days) | Activity (FU/g) | Activity (%) | Activity (FU/g) | Activity (%) |
| 0 | 20,100 | 100 | 20,100 | 100 |
| 50 | 19,100 | 95 | 20,333 | 101 |
| 107 | 13,967 | 69 | 18,167 | 90 |
| 139 | 13,267 | 66 | 19,367 | 96 |
| 170 | 11,933 | 59 | 18,733 | 93 |
| 237 | 8,567 | 43 | 19,044 | 95 |
| 330 | 5,360 | 27 | 17,477 | 87 |

TABLE 4
Undispersed Enzyme in Environmental Test Chamber

| Duration | Enzyme | Relative |
|---|---|---|
| (Days) | Activity (FU/g) | Activity (%) |
| 0 | 265,000 | 100 |
| 27 | 201,667 | 76 |
| 84 | 183,333 | 69 |
| 116 | 192,667 | 73 |
| 147 | 182,667 | 69 |
| 214 | 169,667 | 64 |
| 307 | 156,456 | 59 |

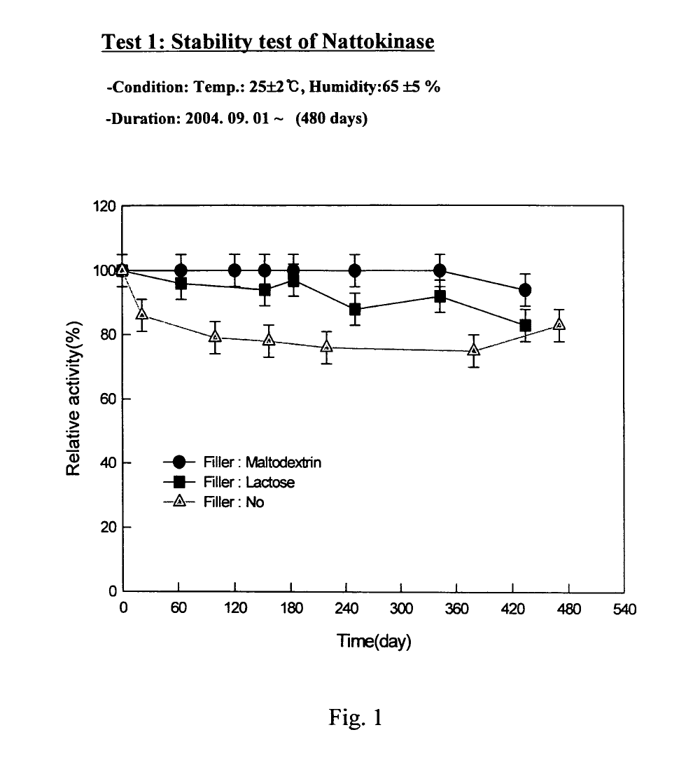

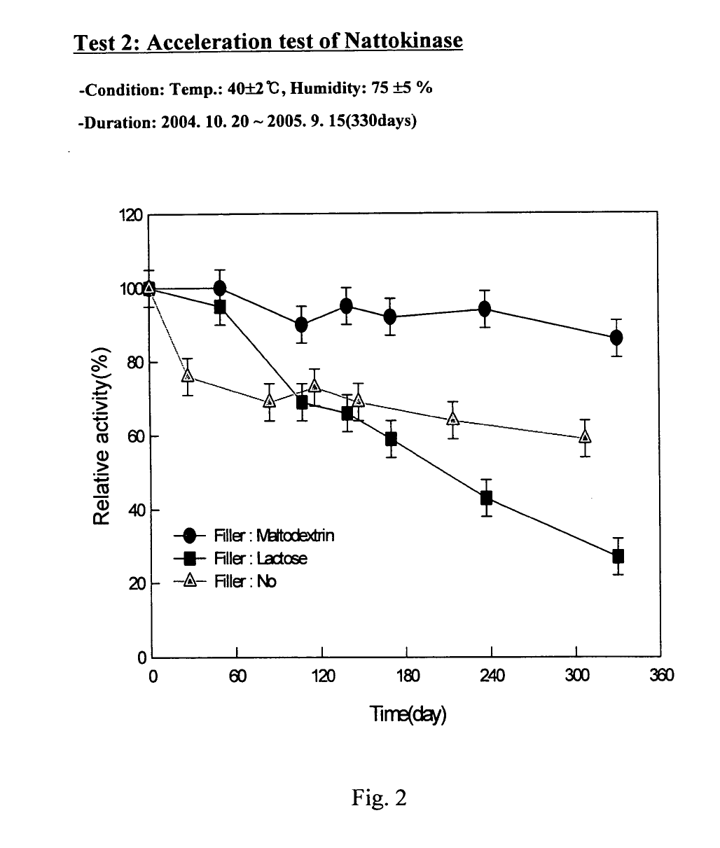

The change in fibrinolytic activity for each sample also is shown in FIGS. 1 and 2 and confirms that, surprisingly, the maltodextrin dispersions of enzyme have improved stability while dispersions of the enzyme in lactose, also a sugar, do not provide improved stability. The maltodextrin dispersion in the standard storage room retained 100% of its original activity through 360 days and showed only a 5% reduction in activity at the 420^{th} day. The lactose dispersion lost 4% of its activity in the first 60 days and by the 250^{th} day lost 12% of its enzymatic activity. By the 434^{th} day, when the maltodextrin dispersion showed its first loss in activity of 5%, the lactose dispersion had lost 17% of its activity. The undispersed nattokinase lost 14% of its activity by the 21^{st} day and by the 99^{th} day had lost 21% of its activity. The assay performed on the 470^{th} day show a loss in activity for the undispersed nattokinase of 26%.

The results for the samples stored in the environmental test cabinet illustrate even more dramatically the stability improvement provided by the maltodextrin dispersion. The maltodextrin/ enzyme dispersion maintained 100% of its activity for 50 days, while the lactose/enzyme dispersion already had lost 5% of its activity during that time. By the 237^{th} day of accelerated testing, the maltodextrin/enzyme dispersion retained 95% of its original activity while the lactose/enzyme dispersion retained only 43% of its original activity. The undispersed enzyme lost almost a quarter of its activity after 27 days and by the 214^{th} day had lost 36% of its activity during accelerated testing.

1. A soluble composition exhibiting enzymatic activity and improved stability, the composition comprising:
(a) dry, powdered enzyme substantially evenly dispersed in
(b) dry, powdered maltodextrin carrier.

2. The composition of claim 1 wherein the enzyme has an average particle size of about 40 mesh and the maltodextrin carrier has an average particle size of about 60 mesh.

3. The composition of claim 1 wherein the enzyme exhibits fibrinolytic activity.

4. The composition of claim 3 wherein the ratio of enzyme to maltodextrin carrier is adjusted to result in a pre-determined level of fibrinolytic activity in the composition.

5. The composition of claim 4 wherein the pre-determined level of fibrinogen activity is about 20,000 FU/g.

6. The composition of claim 3 wherein the fibrinolytic enzyme is nattokinase.

7. A supplement food having enzymatic activity comprising a liquid food component that holds in solution an amount of the composition of claim 1.

8. A supplement food having fibrinolytic activity comprising a liquid food component that holds in solution an amount of the composition of claim 3.

9. A supplement food according to claim 7 wherein the liquid food component is selected from the group consisting of yogurt, milk, water, fruit juice, vegetable juice and mixtures thereof.

10. A supplement food according to claim 8 wherein the liquid food component is selected from the group consisting of yogurt, milk, water, fruit juice, vegetable juice and mixtures thereof.

11. A method for making a soluble composition having enzymatic activity and improved stability comprising:
(a) providing a dry, powdered enzyme; and
(b) substantially evenly dispersing the enzyme in a dry, powdered maltodextrin carrier material by dry mixing.

12. The method of claim 11 wherein the enzyme has an average particle size of about 40 mesh and the maltodextrin carrier material has an average particle size of about 60 mesh.

13. The method of claim 11 wherein the enzyme demonstrates fibrinolytic activity.

14. The method of claim 13 in which the soluble composition has a pre-determined level of fibrinolytic activity, wherein (a) comprises providing a dry, powdered enzyme having a known level of fibrinolytic activity and wherein (b) comprises dispersing the enzyme in the dry, powdered maltodextrin carrier material at an enzyme to maltodextrin ratio sufficient to achieve the pre-determined level of fibrinolytic activity in the soluble composition.

15. The method of claim 14 wherein the pre-determined level of fibrinolytic activity of the soluble composition is about 20,000 FU/g.

16. The method of claim 13 wherein the enzyme is nattokinase.

17. The method of making a supplement food having enzymatic activity comprising dissolving a selected amount of the soluble composition of claim 1 in a liquid food component of the supplement food.

18. The method of making a supplement food having fibrinolytic activity comprising dissolving a selected amount of the soluble composition of claim 3 in a liquid food component of the supplement food.

19. The method of claim 17 wherein the liquid food component is selected from the group consisting of yogurt, milk, water, fruit juices, vegetable juices and mixtures thereof.

20. The method of claim 18 wherein the liquid food component is selected from the group consisting of yogurt, milk, water, fruit juices, vegetable juices and mixtures thereof.

21. The method of treating a subject to increase enzymatic activity in the subject, the method comprising administering to the subject by ingestion a selected amount of the supplement food of claim 7.

22. The method of treating a subject to increase fibrinolytic activity in the subject, the method comprising administering to the subject by ingestion a selected amount of the supplement food of claim 8.
